(19) United States
(12) Patent Application Publication
(10) Pub. No.: US 2026/0215838 A1
(43) Pub. Date: Jul. 30, 2026

(54) ABLATION DEVICE AND TREATMENT METHOD FOR CHOLECYSTITIS

(71) Applicant: OLYMPUS MEDICAL SYSTEMS CORP., Tokyo (JP)

(72) Inventors: Tomofumi KATAYAMA, Kunitachi-shi (JP); Yuji KISHIMOTO, Kodaira-shi (JP)

(73) Assignee: OLYMPUS MEDICAL SYSTEMS CORP., Tokyo (JP)

(21) Appl. No.: 19/574,570

(22) Filed: Mar. 23, 2026

Related U.S. Application Data
(62) Division of application No. 18/115,324, filed on Feb. 28, 2023.
(60) Provisional application No. 63/315,108, filed on Mar. 1, 2022.

(51) Int. Cl. A61B 18/14; A61B 18/00

(52) U.S. Cl. CPC A61B 18/14; A61B 2018/0022; A61B 2018/00589; A61B 2018/00595

(57) ABSTRACT

An ablation device comprises a sheath, a balloon attached to the sheath and being expandable and an electrode located on an outer surface of the sheath. The balloon is located longitudinally adjacent to the electrode.

### RELATED APPLICATION DATA

This application is a divisional application of U.S. Application No. 18/115,324, filed on February 28, 2023, and claims benefit from US Patent Provisional Application No. 63/315,108, filed March 1, 2022, the contents of each of these applications are incorporated herein by reference.

### TECHNICAL FIELD

The present disclosure relates to an ablation device and a treatment method for cholecystitis.

### BACKGROUND

In the bile duct system, which is a luminal organ, the gallbladder is present as a sac-shaped organ.

Acute cholecystitis, which is one of the diseases of the gallbladder, often occurs in a case where calculi formed in the gallbladder move to the cystic duct.

The current standard treatment of acute cholecystitis is laparoscopic cholecystectomy. In laparoscopic cholecystectomy, there is no risk of recurrence because the gallbladder is removed; however, since it is a manual surgical procedure, there is room for improvement in terms of invasiveness.

In the treatment of cholecystitis, a less invasive treatment method than laparoscopic cholecystectomy has been sought. United States Patent No. 8460314 discloses a method of puncturing the gallbladder from the gastrointestinal tract under an ultrasonic endoscope and eliminating the function of the gallbladder mucosa by cauterization or the like. In a case where the function of the gallbladder mucosa is eliminated, bile is not concentrated, and thus the generation and growth of calculi are suppressed.

### SUMMARY

A first aspect of the present disclosure is an ablation device comprises a sheath, a balloon attached to the sheath and being expandable and an electrode located on an outer surface of the sheath. The balloon is located longitudinally adjacent to the electrode.

A second aspect of the present disclosure is a treatment method for cholecystitis comprises inserting an electrode into a cystic duct and using the electrode to electro-cauterize the cystic duct from an inside of the cystic duct.

### BRIEF DESCRIPTION OF DRAWINGS

FIG. 1 is a view showing an example of an operation to access the gallbladder.

FIG. 2 is a view showing an example of an operation to access the gallbladder.

FIG. 3 is a view showing a state in which the gallbladder and the duodenum are communicated with each other through a stent.

FIG. 4 is a view showing an example of the ablation device according to the disclosure.

FIG. 5 is a view showing a process of step B in the treatment method for cholecystitis according to a first embodiment of the disclosure.

FIG. 6 is a view showing a process of step B.

FIG. 7 is a view showing a process of step B according to the second embodiment of the disclosure.

FIG. 8 is a schematic view showing a modified example of the ablation device according to the disclosure.

FIG. 9 is partial enlarged view showing another modified example of the ablation device.

### DETAILED DESCRIPTION

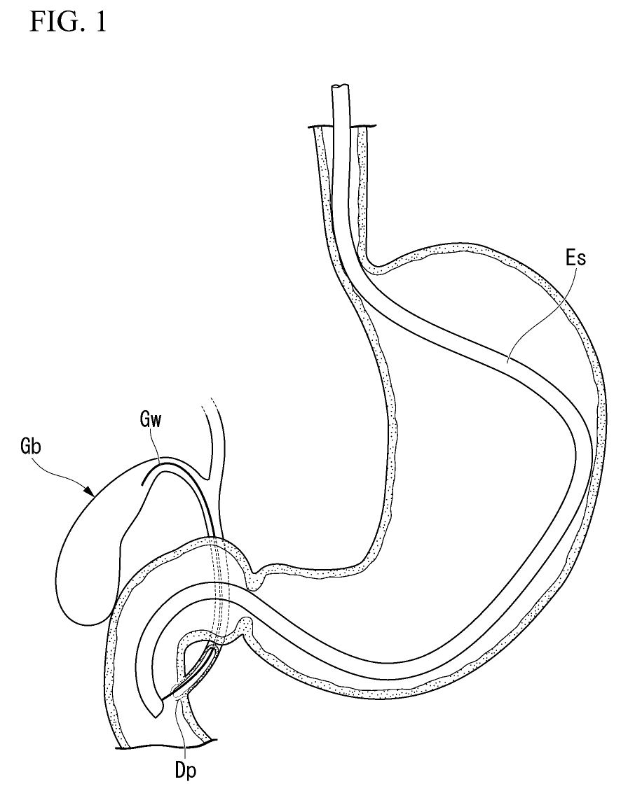

A first embodiment of the method for treatment method for cholecystitis according to the disclosure will be described with reference to FIGS. 1 to 6.

First, the contents of the gallbladder are removed (Step A). The contents of the gallbladder are mainly bile and gallstones (calculi). Bile is removed by gallbladder drainage, and calculi are removed using various treatment tools.

There are roughly two kinds of access routes to the gallbladder for performing gallbladder drainage, and either route may be used in the embodiment.

In an access route via the duodenum papilla (hereinafter, may be referred to as "route A"), as shown in FIG. 1, a guide wire Gw is inserted into the duodenum by the same procedure as used in ERCP (endoscopic retrograde cholangiopancreatography) from an endoscope Es introduced into the duodenal papilla Dp. Then, the distal end of the guide wire Gw is operated to reach the inside of the gallbladder Gb, and a drainage tube is indwelled along the guide wire.

In the transabdominal access route (hereinafter, may be referred to as “route B”), a puncture needle protruding from the endoscope is inserted into the duodenal wall while confirming the position of the gallbladder with an image of the ultrasonic endoscope introduced into the duodenum and further inserted into the gallbladder. Then, in a state in which the puncture needle is inserted into the gallbladder, the guide wire is passed through the puncture needle, the distal end of the guide wire is operated to reach the inside of the gallbladder, and then only the puncture needle is removed with the guide wire being left in place. Then, a drainage tube is indwelled along the indwelled guide wire.

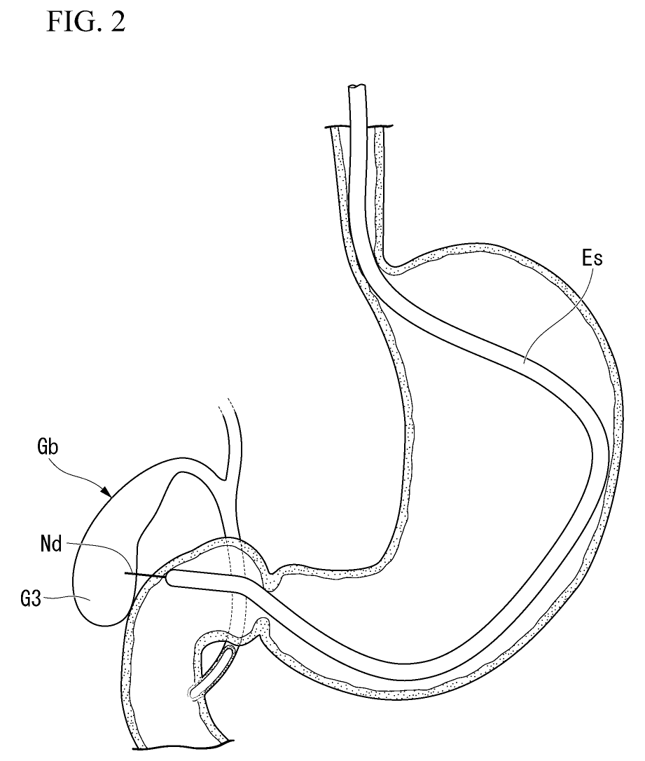

In route B, a drainage route may be established in any one of the cervical part, body part, and bottom part of the gallbladder. However, a drainage route may be established in the bottom part of the gallbladder in consideration of the subsequent procedure described later. In a case where a drainage route is established at the bottom part of the gallbladder, as shown in FIG. 2, a puncture needle Nd protruding from the endoscope Es is inserted into the bottom part G3 of the gallbladder Gb. In a state in which the puncture needle is inserted into the gallbladder, the guide wire is passed through the puncture needle Nd, and when the distal end of the guide wire has reached the inside of the gallbladder, only the puncture needle is removed with the guide wire being left in place.

Due to the above-described treatment, a through-hole communicating the gallbladder and the duodenum is formed.

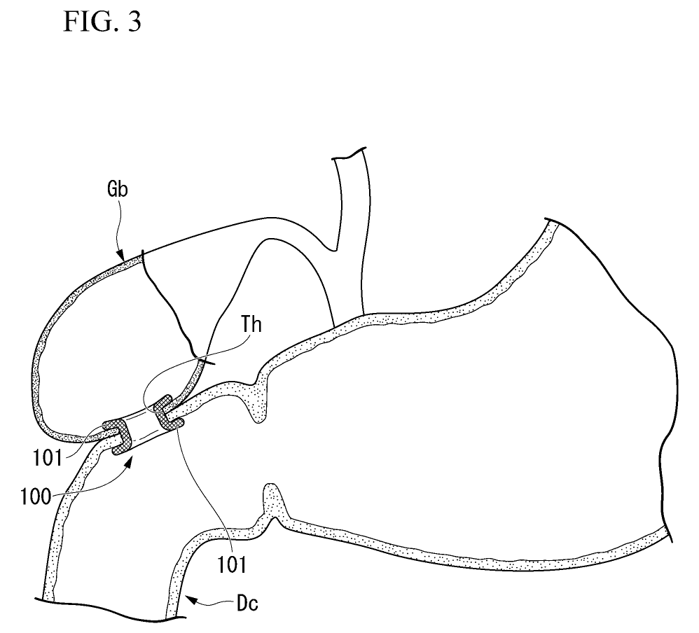

In a case of choosing route B, the distal end of the stent delivery device is operated to reach the gallbladder along the indwelled guide wire, and as shown in FIG. 3, as the drainage tube, a covered stent connecting the gallbladder and the duodenum is indwelled into the through-hole Th formed by the puncture. Thereby the through-hole Th is capable of being opened with a sufficient size and stably maintained in such a state. A covered stent 100 shown in FIG. 3 has flanges 101 on both sides in the axial direction of the tubular shape, and is configured such that it is difficult for the stent 100 to be removed from the gallbladder Gb and the duodenum Dc after indwelling. A covered stent without the flange 101 can also be used. In that case, the operator may suture the covered stent to the gallbladder or duodenum so that the covered stent does not come out of the position.

In a case where there are calculi in the gallbladder after the drainage is complete, the calculi are removed as necessary.

The calculi can be removed by various known methods. Specific examples thereof include removal and crushing of calculi with a basket, removal of calculi by suction with a suction catheter, and crushing of calculi with a laser of a laser irradiation device. The calculi may be removed outside the gallbladder in their original form, or may be removed by making smaller than the original form by crushing or the like.

After the removal of the contents is completed, the inside of the gallbladder may be washed with a treatment liquid or the like including a physiological saline solution or an anti-inflammatory agent.

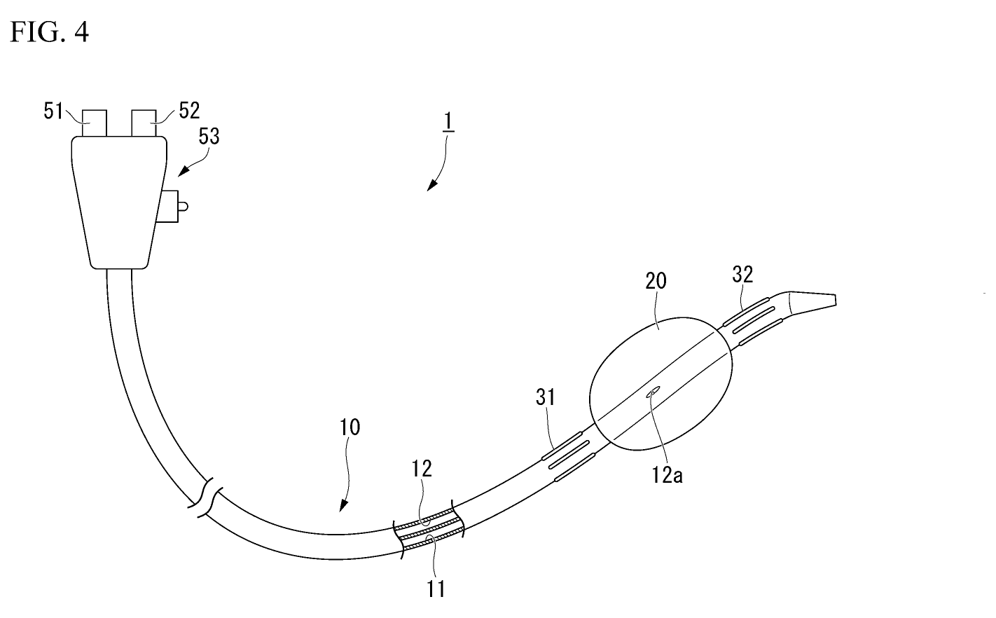

Next, a portion of the gallbladder including the cystic duct is cauterized (Step B). FIG. 4 shows a schematic view of the ablation device (hereinafter, simply referred to as “device”) used for step B. A device 1 as shown in FIG. 4 includes a sheath 10, a balloon 20 connected to the sheath 10, and an electrode (ablation element) exposed on an outer circumferential surface of the sheath 10.

The sheath 10 includes a guide wire lumen 11 opening at both the distal end and the proximal end thereof, and a balloon operation lumen 12 opening at the proximal end and the outer circumferential surface vicinity of the distal end. A proximal end opening of the guide wire lumen 11 is connected to a first port 51. A proximal end opening of the balloon operation lumen 12 is connected to a second port 52.

The balloon 20 is attached around the sheath 10 so as to cover an opening 12a, which is formed on the outer circumferential surface of the sheath 10, of the balloon operation lumen 12. Both the distal end side and the proximal end side of the balloon are sealed and airtightness of the balloon 20 is maintained. Accordingly, the balloon 20 can be expanded by supplying air or a liquid from the second port 52.

A plurality of wire shaped members consisting of a conductor is inserted into an inner wall of the sheath 10. The plurality of wire shaped members is exposed on the outer circumferential surface of the sheath 10 at both a predetermined range in the distal end side from the balloon 20, and a predetermined range in the proximal end side from the balloon 20. Thereby, a first electrode 31 is provided at the proximal side from the balloon 20 and positioned closer to the balloon 20, and a second electrode 32 is provided at the distal side from the balloon 20 and positioned closer to the balloon 20. That is, the device 1 includes two ablation elements, namely the first electrode 31 and the second electrode 32. In other word, the balloon 20 is located longitudinally adjacent to one or more of the first electrode 31 and the second electrode 32. The first electrode 31 and the second electrode 32 may be arranged at above described sealed portion fixing the balloon 20 to the sheath 10.

In the sheath 10, a portion at which the second electrode 32 is provided is curved to the same degree as an average shape of the cystic duct when in a natural state in which no external force is applied to the sheath.

The plurality of the wire shaped members is electrically connected to a plug 53 at the proximal end side of the device 1. Thereby, the device supplies electric power to the first electrode 31 and the second electrode 32 by the power source connected to the plug 53.

In the embodiment, step B is performed via route B.

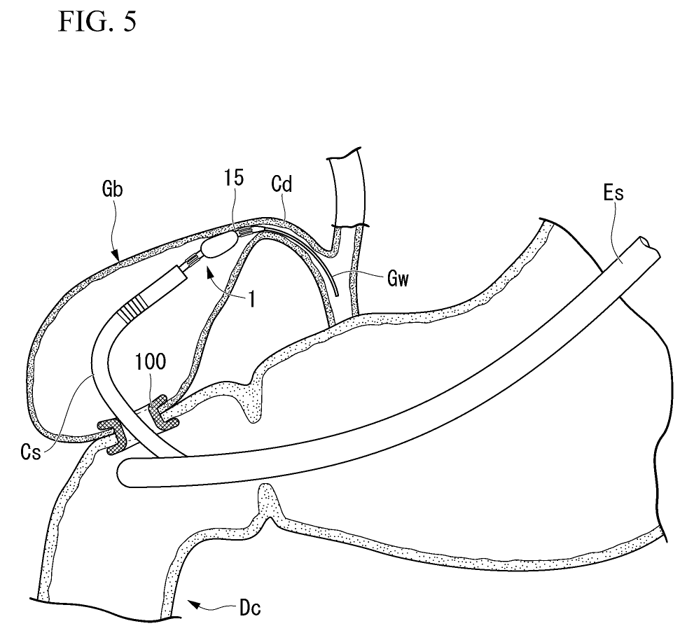

A cholangioscope Cs capable of being bendably operated is protruded from the treatment tool channel of the endoscope Es introduced into the duodenum Dc, thereby inserting it into the gallbladder Gb via the stent 100. Further, the guide wire GW is protruded from the treatment tool channel of the cholangioscope Cs (see FIG. 5) and inserted into the cystic duct Cd from an inside of the gallbladder Gb. The distal end of the guide wire Gw may be inserted be positioned inside the common bile duct or positioned to be protruded from the duodenal papilla through the common bile duct. A distal end position of the guide wire Gw can be confirmed thorough an X-ray fluoroscopic image or the like.

The operator inserts the guide wire Gw into the guide wire lumen 11 from the distal opening of the sheath 10. After that, as shown in FIG. 5, the device 1 is inserted into the cystic duct while being guided by the guide wire Gw. Due to this operation, in the sheath 10, a portion where is the distal end side from the balloon 20 and is provided with the second electrode 32 enters the cystic duct Cd.

Next, the operator supplies fluid from the second port 52 to expand the balloon 20 and advances the device 1 toward the cystic duct Cd. In other word, the operator supplies fluid from the second port 52 to inflating the balloon 20 and advances the device 1 toward the cystic duct Cd.

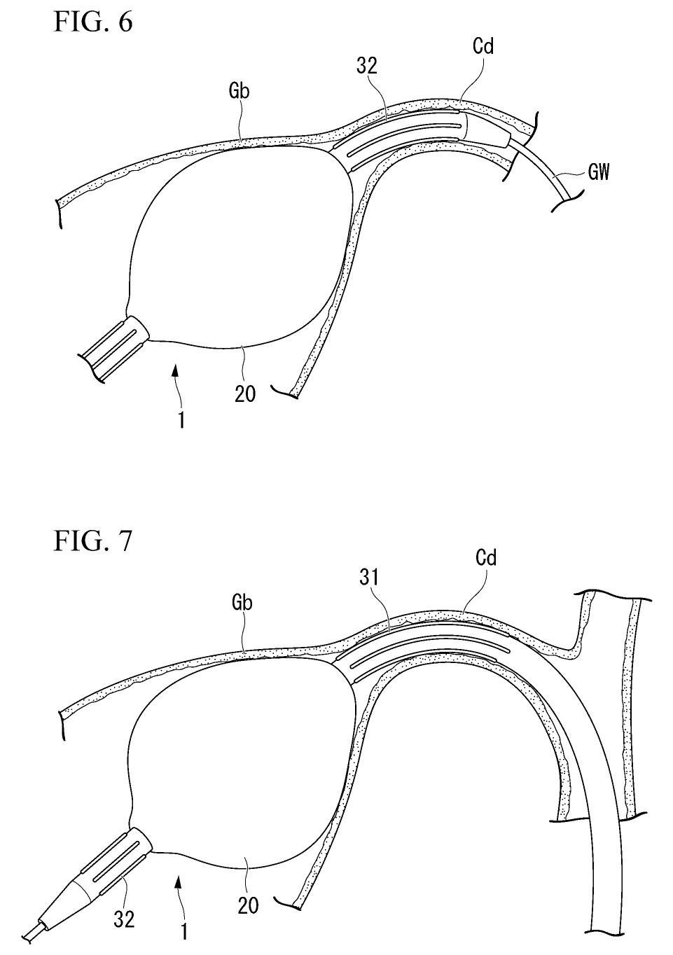

Since the expanded (inflated) balloon 20 cannot enter the cystic duct Cd, as shown in FIG. 6, the balloon 20 is made to be pressed to a border portion between the gallbladder Gb and the cystic duct Cd due to the operation, and at least a portion of the second electrode 32 is arranged inside the cystic duct Cd.

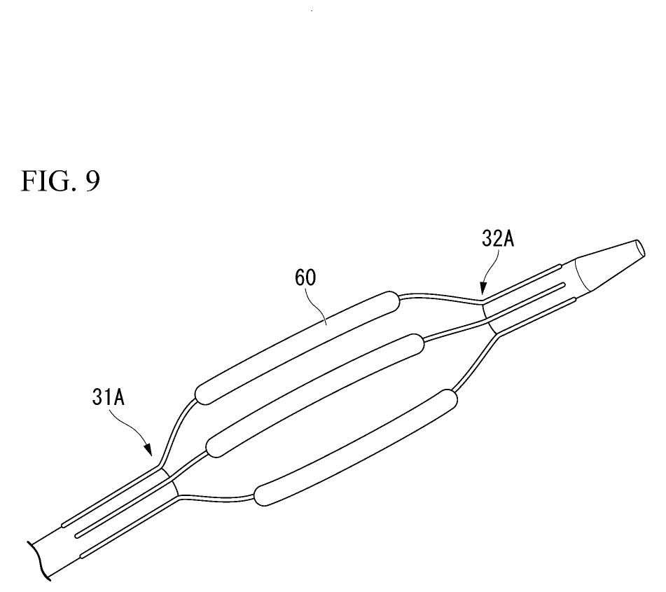

The operator supplies electric power to the second electrode 32 from the plug 53, and cauterizes the cystic duct Cd from an inside thereof. In other word, the operator uses the second electrode 32 to electro-cauterize the cystic duct Cd from an inside of the cystic duct. Since the thickness of the wall of the cystic duct Cd is thin, such as 3 mm or less, the cystic duct Cd is heated as a whole in the thickness direction by the cauterization. Blood vessels such as the arteria cystica supplying nutrients to the gallbladder, and nerves such as the sympathetic nerve or parasympathetic nerve of the gallbladder are elongated toward the bottom part of the gallbladder via an outer tissue of the cystic duct. The blood vessels or the nerves outside or inside the wall of the cystic duct are cauterized by the cauterization of the device and the blood vessels or the nerves are denatured by heat. Accordingly, a function including a bile concentration ability of the gallbladder is largely reduced or lost. In the example shown in FIG. 9 described later, since a portion of the electrode is also exposed on the balloon, the device cauterizes everywhere in the vicinity of the cystic duct, and the blood vessels and the nerves are reliably cauterized. Furthermore, since the cystic duct is cauterized, the tissue becomes pasty and the cystic duct recovers so as to be occluded after repairing.

As described above, step B is completed.

After the completion of step B, the operator removes the device 1 and cleans up any contaminants in both the gallbladder and the cystic duct (Step C).

Furthermore, the operator occludes the cystic duct by using a clip or a thread (Step D).

Finally, the stent 100 is removed, and the access route communicating the duodenal papilla with the gallbladder is occluded (Step E).

As described above, the treatment method for cholecystitis according to the embodiment is completed.

Since a function of the gallbladder is reduced or lost by step B, bile is not concentrated even if it has entered the gallbladder. Therefore, since calculi are neither generated nor grow, a relapse or a recrudescence of cholecystitis is suitably suppressed.

Furthermore, since the cystic duct is occluded by step D, bile flow itself is also suppressed and a possibility of a relapse or a recrudescence of cholecystitis is further suppressed.

As described above, the treatment method for cholecystitis according to the embodiment is capable of suitably suppressing a relapse or a recrudescence of cholecystitis without removing the gallbladder. Accordingly, invasiveness to the patient can be significantly decreased in comparison with laparoscopic cholecystectomy.

In step B, since the balloon 20 is expanded and contacts the border portion between the cystic duct and the gallbladder, the second electrode 32 is easily positioned in the cystic duct, and reliably cauterizes an area including the cystic duct. That is, in step B, the ablation element can be extremely easily positioned using the device 1.

In the treatment method for cholecystitis, not that a function is made to be decrease or lose by directly cauterized the mucosa in the gallbladder, but that a function of the gallbladder is made to be decrease or lose by cauterizing the cystic duct to stop supplying the blood to the gallbladder or stop controlling to the gallbladder. Therefore, in comparison with the technique written in U.S. patent No. 8,460,314, it becomes less invasive and same level effect is achieved with smaller cauterization area.

In step B of the embodiment, the cauterization may be performed in a coagulation mode which causes the tissue not to transpire. In this case, a cauterized tissue is melted and increasing the viscosity, thereby inner walls of the cystic duct are bonded after removing the device 1. As a result, step D is performed when removing the device, and it is not necessary to use other instrument such as a clip.

Either of step C and step D may be performed first. Further, any one of steps C and D or both may be omitted according to the situation.

A second embodiment of the disclosure will be described with reference to FIG. 7. In the following description, the same reference signs are applied to constituent elements common to those which have already been described, and duplicate description will be omitted.

In the embodiment, step B is performed via route A. Step A may be performed via any one of route A and route B.

In step B, as shown in FIG. 1, the operator introduces the device 1 to the gallbladder Gb from the treatment tool channel of the endoscope Es introduced into the duodenal papilla Dp while being guided by the guide wire Gw inserted into the gallbladder Gb from the duodenum Dc via the cystic duct. At the time, the operator advances the device 1 to a position where the balloon 20 is fully positioned inside the gallbladder while confirming the position with an X-ray fluoroscopic image or an image of the ultrasonic endoscope.

Subsequently, the operator expands the balloon 20 and then removes the device. Due to the operation, as shown FIG. 7, the balloon 20 is pressed to the border portion between the gallbladder Gb and the cystic duct Cd, and at least a portion of the first electrode 31 is arranged inside the cystic duct Cd.

After that, the operator supplies electric power to the first electrode 31 from the plug 53, and cauterizes the cystic duct Cd from an inside thereof. Step B is completed.

Subsequently, although steps C and D are performed same as the first embodiment, in a case of the embodiment, step C may be performed prior to step D.

In the embodiment, since step D is performed also serves as step E, it is not necessary to independently perform step E.

The treatment method according to the embodiment is also capable of suitably suppressing a relapse or a recrudescence of cholecystitis without removing the gallbladder same as the first embodiment.

Each of the embodiment of the present disclosure has been described above. However, the technical scope of the present disclosure is not limited to the above embodiment, and it is possible to change the combination of constitutional elements, make various changes to each constitutional element, or delete a constitutional element without departing from the gist of the present disclosure. Some additional changes are exemplified; however, these are not all, other changes are possible. Two or more of these changes may be combined as appropriate, or they may be combined with the above-described changes.

The ablation device used in the disclosure does not limited to the device 1 has been described above, it may be modified in various ways. A part of that will be described below.

Any one of the first electrode and the second electrode are provided, and the device is dedicated for route A or route B. In a case the second electrode does not include, it is not necessary elongate the sheath to the distal end side of the balloon.

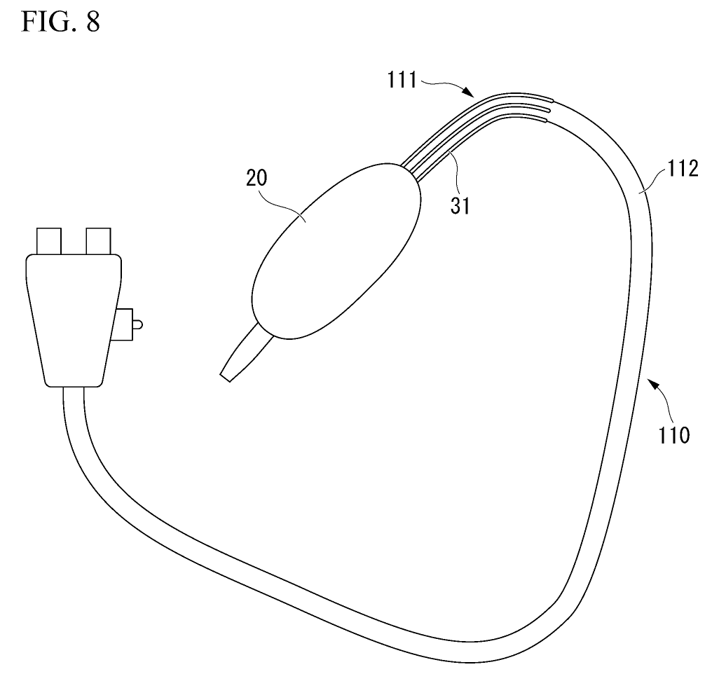

The sheath may have a curvature tendency, and the sheath is made to be curved same as a general shapes of the cystic duct and the common bile duct from the duodenal papilla to the gallbladder when the sheath protrudes from the endoscope or the like. In this case, the sheath is positioned at the proximal side of the balloon 20 like the sheath 110 of the modified example as shown in FIG. 8, the sheath has a first curved portion 111 having a curvature shape corresponding to the cystic duct, and a second curved portion 112 provided continuously to the first curved portion 111 and having a curvature shape corresponding to the common bile duct and the branch portion toward the cystic duct. The first electrode 31 is provided from the first curved portion 111 to a vicinity of the balloon 20, and at least a portion of the first electrode 31 is arranged between the balloon 20 and the first curved portion 111. The first electrode 31 may be arranged at a whole of the first curved portion 111.

This modification is effective in a case in which the ablation device is constituted to correspond to route A.

The electrode may elongate on the balloon 20 like the first electrode 31A and the second electrode 32 A of the modified example as shown FIG. 9. According to the configuration, in step B, since the cervical part, which is positioned closer to the cystic duct, is also capable of being cauterized, a function of the gallbladder can be made to be more decrease. At this time, as shown in FIG. 9, unnecessary area in the electrode positioned on the balloon 20 may allow to be covered by the insulation coating 60.

Next, a part of the changeable modification of the treatment method for cholecystitis according to the disclosure is exemplified below.

All steps according to the disclosure may be performed via a transcutaneous access route. In the transcutaneous access route, the abdominal wall is punctured while confirming the position of the gallbladder with an X-ray fluoroscopic image, and a puncture needle is inserted into the gallbladder via the liver parenchyma, thereby the transcutaneous access route to the gallbladder is established. When closing the access route in step E, a clip or an adhesive agent or the like may be used.

In the treatment method according to the disclosure, all steps are not necessary to be performed via the same access route. Therefore, the treatment method for cholecystitis according to the disclosure is performed via a route appropriately combining of two or more of route A, route B, and the transcutaneous access route.

What is claimed is:

1. A treatment method for cholecystitis, comprising:
inserting an electrode into a cystic duct; and
using the electrode to electro-cauterize the cystic duct from an inside of the cystic duct.

2. A treatment method for cholecystitis, comprising:
inserting an electrode of an ablation device into a cystic duct; and
using the electrode to electro-cauterize the cystic duct from an inside of the cystic duct,
wherein the ablation device includes:
a sheath,
a balloon attached to the sheath and being expandable, and
the electrode located on an outer surface of the sheath,
wherein the balloon is located longitudinally adjacent to the electrode, and
wherein the method further comprises using the balloon to position the electrode in the cystic duct.

3. The treatment method for cholecystitis according to claim 2, wherein using the balloon to position the electrode in the cystic duct includes inflating the balloon.

4. The treatment method for cholecystitis according to claim 3, wherein using the balloon to position the electrode in the cystic duct further includes pressing the inflated balloon to a boundary portion of the cystic duct and a gallbladder.

5. The treatment method for cholecystitis according to claim 4, wherein a distal end of the inflated balloon contacts the boundary portion of the cystic duct and the gallbladder.

6. The treatment method for cholecystitis according to claim 3, wherein using the balloon to position the electrode in the cystic duct further includes pulling the inflated balloon to a boundary portion of the cystic duct and a gallbladder.

7. The treatment method for cholecystitis according to claim 6, wherein a proximal end of the inflated balloon contacts the boundary portion of the cystic duct and the gallbladder.

8. The treatment method for cholecystitis according to claim 1, wherein using the electrode to electro-cauterize the cystic duct further includes coagulation to prevent transpiration from a tissue of the cystic duct.

9. The treatment method for cholecystitis according to claim 1, further comprising removing a content of a gallbladder before using the electrode to electro-cauterize the cystic duct.

10. The treatment method for cholecystitis according to claim 1, further comprising closing the cystic duct after using the electrode to electro-cauterize the cystic duct.

11. The treatment method for cholecystitis according to claim 2, wherein using the electrode to electro-cauterize the cystic duct further includes coagulation to prevent transpiration from a tissue of the cystic duct.

12. The treatment method for cholecystitis according to claim 2, further comprising closing the cystic duct after using the electrode to electro-cauterize the cystic duct.
